(19) United States
(12) Patent Application Publication
(10) Pub. No.: US 2024/0262767 A1
(43) Pub. Date: Aug. 8, 2024

(54) RECYCLED CONTENT NEOPENTYL GLYCOL

(71) Applicant: Eastman Chemical Company, Kingsport, TN (US)

(72) Inventors: Daryl Bitting, Longview, TX (US); David Eugene Slivensky, Tatum, TX (US); Xianchun Wu, Longview, TX (US)

(73) Assignee: Eastman Chemical Company, Kingsport, TN (US)

(21) Appl. No.: 18/567,801

(22) PCT Filed: Jun. 8, 2022
(86) PCT No.: PCT/US2022/032600

§ 371 Date: Dec. 7, 2023

Related U.S. Application Data
(60) Provisional application No. 63/202,451, filed on Jun. 11, 2021.

(51) Int. Cl. C07C 29/141; C07C 4/02; C07C 29/151; C07C 45/50; C07C 45/64

(52) U.S. Cl. CPC C07C 29/141; C07C 4/02; C07C 29/1516; C07C 45/50; C07C 45/64

(57) ABSTRACT

Recycled content neopentyl glycol (r-NPG) is produced using a process and system that applies physical and/or credit-based recycled content from one or more feed materials to the NRG produced from the feed materials.

### BACKGROUND

Neopentyl glycol (2,2-dimethylpropane-1,3-diol) is an important chemical used in a wide variety of applications. In particular, it is used as an intermediate in forming various types of materials, including, but not limited to, paints, coatings, lubricants, and plasticizers. Additionally, neopentyl glycol can be used in the synthesis of polyesters to enhance stability and other desirable properties.

The demand for recycled chemical products continues to grow, but there is no clear path to recycled neopentyl glycol through mechanical recycling. Thus, there exists a need for a commercial process to produce recycled content neopentyl glycol.

### SUMMARY

In one aspect, the present technology concerns a process for producing neopentyl glycol having recycled content, the process comprising: (a) hydroformylating a first propylene and a first syngas to thereby produce an isobutyraldehyde; (b) aldol condensing at least a portion of the isobutyraldehyde and a first formaldehyde to thereby produce a hydroxypivaldehyde; and (c) hydrogenating at least portion of the hydroxypivaldehyde with a first hydrogen to thereby produce a neopentyl glycol (NPG), wherein the NPG comprises recycled content from one or more of the following source materials—(i) a waste plastic, (ii) a recycled content syngas (r-syngas), (iii) a recycled content formaldehyde (r-formaldehyde), (iv) a recycled content hydrogen (r-H2), and/or (v) a recycled content propylene (r-propylene).

In one aspect, the present technology concerns a process for producing neopentyl glycol having recycled content, the process comprising: (a) aldol condensing a first isobutyraldehyde and a first formaldehyde to thereby produce a hydroxypivaldehyde; and (b) hydrogenating at least portion of the hydroxypivaldehyde with a first hydrogen to thereby produce a neopentyl glycol (NPG), wherein the NPG comprises recycled content from one or more of the following source materials—(i) a waste plastic, (ii) a recycled content formaldehyde (r-formaldehyde), and/or (iii) a recycled content hydrogen (r-H2).

In one aspect, the present technology concerns a process for producing formaldehyde having recycled content, the process comprising: (a) carbon reforming a hydrocarbon-containing feedstock to provide a first syngas; (b) synthesizing a first methanol from at least a portion of the first syngas; and (c) dehydrogenating at least a portion of the first methanol to provide formaldehyde, wherein the formaldehyde comprises recycled content from one or more of the following source materials—(i) a waste plastic, (ii) a recycled content hydrocarbon-containing feedstock (r-HC), (iii) a recycled content syngas (r-syngas), and/or (iv) a recycled content methanol (r-methanol).

### BRIEF DESCRIPTION OF THE DRAWINGS

FIG. 1 is a block flow diagram illustrating the main steps of a process and facility for making recycled content neopentyl glycol (r-NPG) and, optionally, a recycled content formaldehyde (r-formaldehyde), where the r-NPG (and the r-formaldehyde) has physical recycled content from one or more source materials;

FIG. 2 is a block flow diagram illustrating the main steps of a process and facility for making recycled content neopentyl glycol (r-NPG) and, optionally, a recycled content formaldehyde (r-formaldehyde), where the r-NPG (and the r-formaldehyde) has credit-based recycled content from one or more source materials; and

FIG. 3 is a block flow diagram illustrating the main steps of a process and facility for making recycled content neopentyl glycol (r-NPG) and, optionally, a recycled content formaldehyde (r-formaldehyde), where the r-NPG (and the r-formaldehyde) has both physical and credit-based recycled content from one or more source materials.

FIG. 4 is a block flow diagram similar to FIG. 3, but showing that the NPG production process can start at aldol condensation, with isobutyraldehyde and/or r-isobutyraldehyde being supplied from a different location and/or a different source.

### DETAILED DESCRIPTION

We have discovered new methods and systems for producing neopentyl glycol and, optionally, formaldehyde having recycled content. More specifically, we have discovered a process and system for producing NPG where recycled content from waste materials, such as waste plastic, are applied to NPG (or formaldehyde) in a manner that promotes the recycling of waste plastic and provides NPG (or formaldehyde) with substantial amounts of recycled content.

In general, NPG can be formed by first hydroformylating syngas and propylene to form isobutyraldehyde. At least a portion of the isobutyraldehyde can be subjected to aldol condensation (e.g., aldol condensed) with formaldehyde to produce the intermediate hydroxypivaldehyde. The hydroxypivaldehyde can then be converted to neopentyl glycol (NPG), either by hydrogenation or by reaction with a stoichiometric excess of formaldehyde. In some embodiments, the formaldehyde used to form the hydroxypivaldehyde (or NPG) can be formed by reacting syngas to form methanol, then dehydrating the methanol to produce formaldehyde.

The NPG (or formaldehyde) produced at the facility can include recycled content from one or more source materials including, for example, waste plastic, recycled content syngas (r-syngas), recycled content hydrogen (r-H2), recycled content hydrocarbon-containing feed (r-HC feed), recycled content propylene (r-propylene) and recycled content formaldehyde (r-formaldehyde). The recycled content in the NPG (or the formaldehyde) can be physical and may directly originate from at least one of these streams, and/or the recycled content may be credit-based and applied to a target stream in the NPG (or formaldehyde) process from one or more of these source streams.

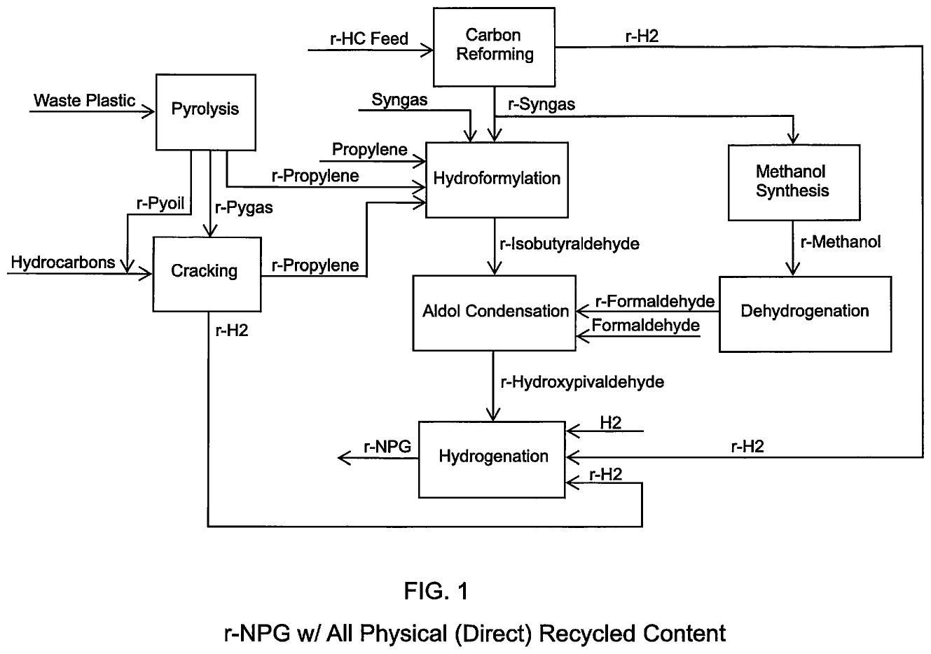

Turning now to FIG. 1, one embodiment of a process and facility for forming NPG with physical (direct) recycled content is provided. The recycled content in the NPG can originate from the pyrolysis of waste plastic (and/or cracking of pyrolyzed waste plastic materials) and/or from carbon reforming of a recycled content hydrocarbon-containing feed (r-HC feed). The resulting NPG can have a total recycled content of at least 5, at least 10, at least 25, at least 50, or at least 65 percent and/or less than 100, less than 99, less than 95, less than 90, or less than 85 percent.

As shown in FIG. 1, waste plastic can be pyrolyzed to form recycled content pyrolysis gas (r-pygas) and recycled content pyrolysis oil (r-pyoil). All or a part of the r-pygas and/or r-pyoil can be introduced into a cracking facility, for example as shown (it can also be sent to FCC to make propylene), where it can be used to produce a recycled content olefin, such as recycled content propylene (r-propylene). Alternatively, or in addition, recycled content propylene may also be separated out in the pyrolysis facility. The feed to the cracking facility can include only r-pyoil and/or r-pygas, or it can also include non-recycled content hydrocarbon, such as naphtha and others (e.g., C5 to C22) or lighter hydrocarbon components (e.g., C2 to C5).

As shown in FIG. 1, all or a portion of the recycled content propylene from the pyrolysis and/or cracking facilities can be subjected to hydroformylation with syngas to form recycled content isobutyraldehyde (r-isobutyraldehyde). In one or more embodiments, at least a portion of the propylene fed to the hydroformylation can be non-recycled content propylene. In some embodiments, the propylene used in hydroformylation can include at least 50, at least 75, at least 90, or 100 percent recycled content.

In some embodiments, the production facility may also include carbon reforming of a recycled content hydrocarbon-containing feed stream (r-HC Feed) to produce a recycled content syngas (r-syngas) and a recycled content hydrogen (r-H2) each having physical recycled content. In some embodiments, the feed to carbon reforming can comprise both a recycled content feed component (e.g., waste plastic) and a non-recycled content feed component (e.g., coal, a liquid hydrocarbon, and/or a gaseous hydrocarbon). In one embodiment, the carbon reforming is partial oxidation gasification that is fed with coal and waste plastic. In another embodiment, the carbon reforming is plasma gasification of a predominately waste plastic feed. In yet another embodiment, the carbon reforming is partial oxidation gasification fed with a non-recycled content liquid or gaseous hydrocarbon and a recycled content pyrolysis oil produced from the pyrolysis of waste plastic. In some embodiments, the carbon reforming can include catalytic reforming, while in other embodiments, the carbon reforming can include steam reforming.

In some embodiments, all or a portion of the r-syngas from carbon reforming may be used in hydroformylation. In some cases, both the propylene and syngas used in hydroformylating can have recycled content, while in some cases, one or both of the propylene and syngas may have non-recycled content. At least some of the syngas used in hydroformylation may include non-recycled content. In some embodiments, the syngas used in hydroformylation can include at least 50, at least 75, at least 90, or 100 percent recycled content.

Additionally, as shown in FIG. 1, at least a portion of the r-syngas from carbon reforming can be reacted in the presence of a catalyst to form recycled content methanol (r-methanol). The r-methanol can then be dehydrated to form recycled content formaldehyde (r-formaldehyde). At least a portion or all of the recycled content formaldehyde can then be condensed with the r-isobutyraldehyde as shown in FIG. 1 to produce recycled content hydroxypivaldehyde (r-hydroxypivaldehyde). In some embodiments, at least a portion of the formaldehyde used to form the hydroxypivaldehyde may be non-recycled content formaldehyde. In some embodiments, the formaldehyde used to form hydroxypivaldehyde can include at least 50, at least 75, at least 90, or 100 percent recycled content. Similarly, in some embodiments, at least a portion of the methanol used to form the formaldehyde may be non-recycled content methanol (not shown in FIG. 1). In some embodiments, the methanol used to form the formaldehyde can include at least 50, at least 75, at least 90, or 100 percent recycled content.

As shown in FIG. 1, the r-hydroxypivaldehyde formed by aldol condensation in the NPG production facility can then be hydrogenated to produce recycled content neopentyl glycol (r-NPG). In some embodiments, at least a portion of the hydrogen used in hydrogenation can be recycled content hydrogen (r-H2) and may originate from, for example, the carbon reforming of the r-HC feed and/or from the cracking of pyrolyzed waste plastic (e.g., r-pygas and/or r-pyoil), or from electrolysis of water by renewable electricity. When originating from the cracking facility, the hydrogen stream may be withdrawn after the final compression stage such as, for example, in the cold box or from a demethanizer column overhead.

The resulting stream of r-NPG can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. Similarly, the r-formaldehyde can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent.

The amount of physical recycled content in the r-NPG can determined by tracing the amount of recycled material along a chemical pathway starting with waste plastic and ending with the NPG. The chemical pathway includes all chemical reactions and other processing steps (e.g., separations) between the starting material (e.g., waste plastic) and the NPG. In FIG. 1, the chemical pathway can include one or more of pyrolysis, cracking, carbon reforming, hydroformylation, aldol condensation, hydrogenation for NPG. The chemical pathway for formaldehyde can include carbon reforming, methanol synthesis, and oxidation and/or dehydrogenation.

In one or more embodiments, a conversion factor can be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of recycled content applied to the r-NPG or r-formaldehyde can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-NPG and/or r-formaldehyde.

The r-NPG can include recycled content from the r-syngas. The chemical pathway can include, for example, carbon reforming, hydroformylation, aldol condensation, and hydrogenation. Alternatively, or in addition, the chemical pathway can include carbon reforming, methanol synthesis, oxidation and/or dehydrogenation, aldol condensation, and hydrogenation. The final r-NPG product can comprise 5 to 5759, 10 to 50, 20 to 40, or 25 to 35 percent recycled content originating from the r-syngas, which can be physical recycled content, credit-based recycled content, or a combination of physical and credit-based recycled content.

The r-NPG can include recycled content from the r-formaldehyde. The chemical pathway can include, for example, carbon reforming, methanol synthesis, oxidation and/or dehydrogenation, aldol condensation, and hydrogenation. The final r-NPG product can comprise 5 to 29, 10 to 29, 20 to 29, or 25 to 29 percent recycled content originating from the r-formaldehyde. The recycled content from the r-formaldehyde can be physical recycled content, credit-based recycled content, or a combination of physical and credit-based recycled content.

The r-NPG can comprise recycled content from the r-H2. The chemical pathway can include, for example, carbon reforming and hydrogenation. The final r-NPG product can comprise 0.1 to 6, 0.5 to 5.5, 1 to 5, or 1.5 to 4.5 percent recycled content from the r-H2. The recycled content from the r-H2 can be physical recycled content, credit-based recycled content, or a combination of physical and credit-based recycled content.

The r-NPG can comprise recycled content from the r-propylene. The chemical pathway can include, for example, pyrolysis, cracking, hydroformylation, aldol condensation, and hydrogenation. The final r-NPG product can comprise 5 to 40, 10 to 40, 20 to 35, or 25 to 32 percent recycled content from the r-propylene. The recycled content from the r-propylene can be physical recycled content, credit-based recycled content, or a combination of physical and credit-based recycled content.

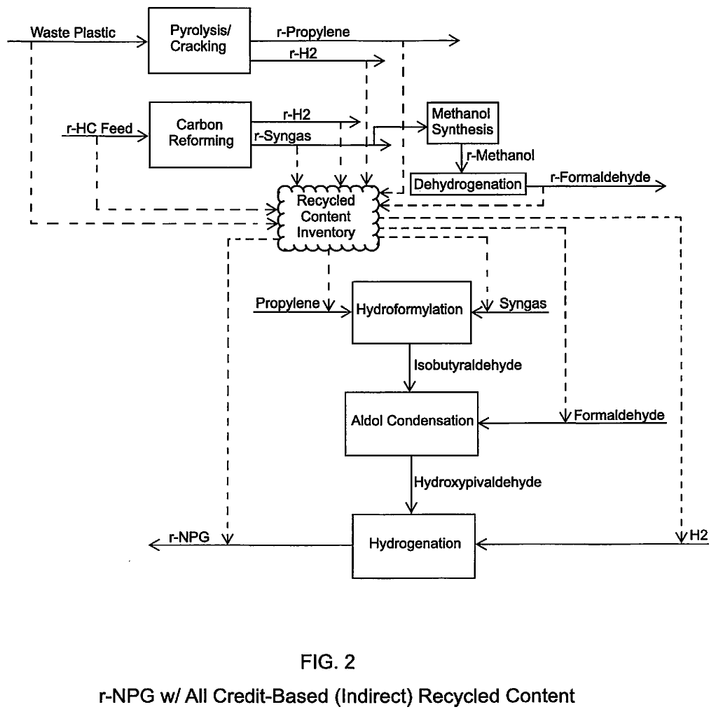

Turning now to FIG. 2, an embodiment where the r-NPG has no physical recycled content, but has credit-based recycled content, is provided. In the process and system depicted in FIG. 2, the r-propylene and r-syngas are not fed directly into the hydroformylation, nor is the r-formaldehyde fed directly into the aldol condensation. Additionally, r-H2 may not be directly used in the hydrogenation in the embodiment shown in FIG. 2.

Instead, recycled content credits from the recycled content streams shown in FIG. 2 (e.g., the r-propylene, the r-H2, the r-syngas, and the r-formaldehyde can be attributed to one or more streams within the production facility. For example, the recycled content credits from one or more of the above streams can be attributed to the syngas and/or propylene fed to the hydroformylation and/or to the formaldehyde fed to the aldol condensation. Further, recycled content credits from one or more of these streams may also be attributed to the hydrogen used to hydrogenate the hydroxypivaldehyde. As such, the r-propylene, the r-H2, the r-syngas, and the r-formaldehyde each act as a “source material” of recycled content credits, and the propylene and syngas fed to the hydroformylation, the formaldehyde fed to the aldol condensation, and the hydrogen fed to the hydrogenation each act as a “target material” to which the recycled content credits are attributed.

In one or more embodiments, the source material has physical recycled content and the target material has less than 100 percent physical recycled content. For example, the source material can have at least 10, at least 25, at least 50, at least 75, at least 90, at least 99, or 100 percent physical recycled content and/or the target material can have less than 100, less than 99, less than 90, less than 75, less than 50, less than 25, less than 10, or less than 1 percent physical recycled content.

The ability to attribute recycled content credits from a source material to a target material removes the co-location requirement for the facility making the source material (with physical recycled content) and the facility making the NPG (or formaldehyde). This allows a chemical recycling facility/site in one location to process waste material into one or more recycled content source materials and then apply recycled content credits from those source materials to one or more target materials being processed in existing commercial facilities located remotely from the chemical recycling facility/site. Further, the use of recycled content credits allows different entities to produce the source material and the r-NPG (or r-formaldehyde). This allows efficient use of existing commercial assets to produce r-NPG (or r-formaldehyde). In one or more embodiments, the source material is made at a facility/site that is at least 0.1, at least 0.5, at least 1, at least 5, at least 10, at least 50, at least 100, at least 500, at least 1,000 or at least 10,000 miles from the facility/site where the target material is used to make NPG or formaldehyde.

The attributing of recycled content credits from the source material (e.g., the r-propylene produced from pyrolysis/cracking of waste plastic) to the target material (e.g., the propylene fed to hydroformylation) can be accomplished by transferring recycled content credits directly from the source material to the target material. Alternatively, as shown in FIG. 2, recycled content credits can be applied from any of the waste plastic, the recycled content hydrocarbon feed to the carbon reforming step (r-HC Feed), the r-syngas, the r-H2, the r-propylene, and/or the r-formaldehyde to the NPG via a recycled content inventory. The recycled content inventory can be a digital inventory or database used to record and track recycled content for various materials at various sites over various time periods.

When a recycled content inventory is used, recycled content credits from the source material having physical recycled content (e.g., the waste plastic, the r-HC Feed, the r-propylene, the r-syngas, the r-H2, and/or the r-formaldehyde in FIG. 2) are booked into the recycled content inventory. The recycled content inventory can also contain recycled content credits from other sources and from other time periods. In one embodiment, recycled content credits in the recycled content inventory can only be assigned to target materials having the same or similar composition as the source materials. For example, as shown in FIG. 2, recycled content credits booked into the recycled content inventory from the r-propylene from pyrolysis/cracking can be assigned to the propylene fed into hydroformylation because the two propylene streams have the same or similar compositions. However, recycled content credits from r-propylene could not be assigned to the formaldehyde fed to aldol condensation because the source and target materials would not be the same or similar.

In some embodiments, all or a portion of the recycled content credit can be applied to one or more target materials (e.g., syngas) upon receipt of one or more waste plastic containing materials at the facility. That is, the waste plastic (or recycled content hydrocarbon feed) need not be processed before applying the credit-based recycled content to the target material. Instead, receipt of the waste plastic (or waste-plastic containing material) at the facility can permit application of recycled content credit to one or more target materials. In most cases, however, such waste plastic will then be processed at the facility within 30, 60, or 90 days to produce one or more of the target materials.

Once recycled content credits have been attributed to the target material (e.g., the syngas, the propylene, or the formaldehyde), the amount of the credit-based recycled content allocated to the NPG is calculated by tracing the recycled content along the chemical pathway from the target material to the NPG. The chemical pathway includes all chemical reactions and other processing steps (e.g., separations) between the target material and the NPG, and a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway.

As with the physical recycled content, the amount of credit-based recycled content applied to the r-NPG can be determined using one of variety of methods, such as mass balance, for quantifying, tracking, and allocating recycled content among various materials in various processes. In certain embodiments the method of quantifying, tracking, and allocating recycled content is overseen by a certification entity that confirms the accuracy of the method and provides certification for the application of recycled content to the r-NPG.

The r-NPG can have 25 to 90, 40 to 80, or 55 to 65 percent credit-based recycled content and less than 50, less than 25, less than 10, less than 5, or less than 1 percent physical recycled content. In certain embodiments, the r-NPG can have 10 to 80, 20 to 75, or 25 to 70 percent credit-based recycled content from one or more of the r-syngas, the r-H2, the r-propylene, and r-formaldehyde, individually.

In one or more embodiments, the recycled content of the r-NPG product (or r-formaldehyde product) can include both physical recycled content and credit-based recycled content. For example, the r-NPG (or r-formaldehyde) can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

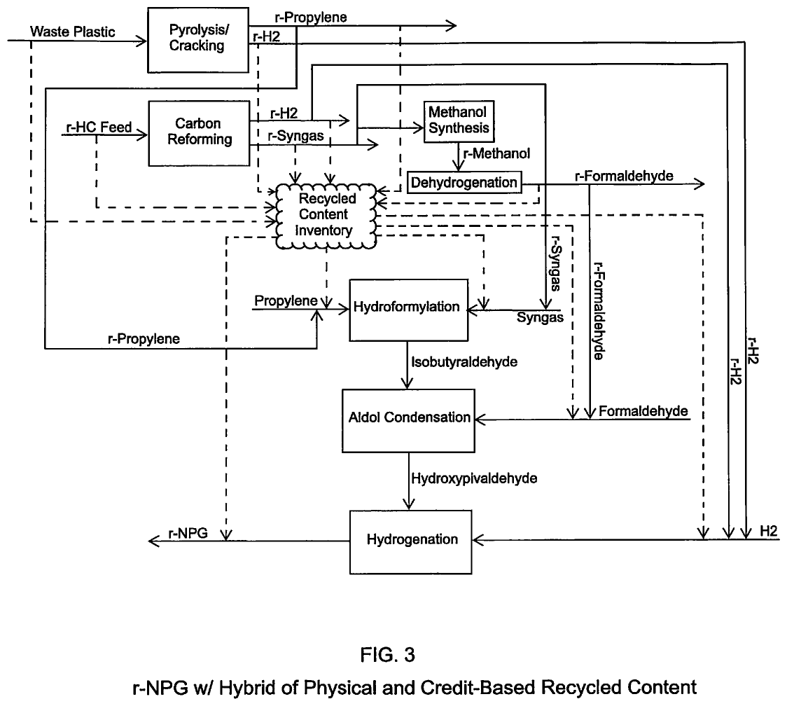

FIG. 3 illustrates several embodiments of an r-NPG (or r-formaldehyde) production process and system, wherein physical recycled content and credit-based recycled content are attributed to the r-NPG (or r-formaldehyde). Any combination of physical (solid lines) and credit-based (dashed lines) recycled content shown in FIG. 3 can be used to form and/or can be attributed to NPG (or formaldehyde) to thereby produce r-NPG (or r-formaldehyde). For example, physical recycled content can be supplied by at least 1, at least 2, at least 3, at least 4, at least 5, or all of the sources shown in FIG. 3, including the waste plastic, the r-HC feed, the r-propylene, the r-H2, the r-syngas, and/or the r-formaldehyde, while the credit-based recycled content can be supplied by one or more of the other sources shown in FIG. 3. In some embodiments, the r-NPG (or r-formaldehyde) can include 10 to 60, 20 to 50, or 25 to 40 percent physical recycled content and 10 to 60, 20 to 50, or 25 to 40 percent credit-based recycled content. Alternatively, the r-NPG (or r-formaldehyde) can include less than 15, less than 10, or less than 5 percent physical recycled content (or credit-based recycled content) and at least 85, at least 90, or at least 95 percent credit based recycled content (or physical recycled content).

For example, in some embodiments, physical recycled content can be provided by r-propylene and/or r-syngas fed to hydroformylation, while credit-based recycled content can be provided by r-formaldehyde. In other embodiments, physical recycled content can be provided by r-formaldehyde, while credit-based recycled content can be provided by r-propylene and/or r-syngas. Alternatively, each of the r-propylene, r-syngas, and r-formaldehyde can be the source of physical recycled content, or each can provide credit-based recycled content to one or more process streams in the NPG production facility.

Turning again to the embodiment shown in FIG. 1, the carbon reforming facility and/or the pyrolysis/cracking facility can be co-located with the r-NPG production facility and/or the r-formaldehyde production steps. In the embodiment shown in FIG. 2, the carbon reforming facility and/or the pyrolysis cracking facility can be remotely located from the r-NPG production facility and/or the r-formaldehyde production steps. In the embodiment shown in FIG. 3, the portions of the pyrolysis/cracking facility providing physical recycled content to the r-NPG (or r-formaldehyde) facility may be co-located, while the portions of the pyrolysis/cracking facility providing credit-based recycled content to the r-NPG (or r-formaldehyde) facility may be remotely located. When remotely located, the two facilities can be at least 0.5, 1, 5, 10, 100, 500, 1000, or 10,000 miles from the other. When co-located, the two facilities can be within 5, 1, 0.5, or 0.25 miles of one another. As discussed previously, when remotely located, two or more of the facilities may be owned and/or operated by the same or by different commercial entities.

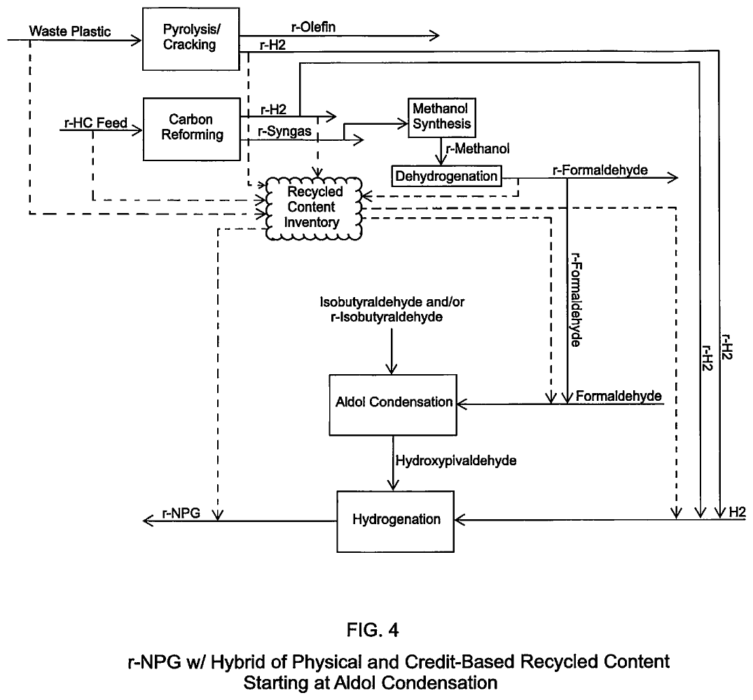

FIG. 4 illustrates a processes and facility similar to that the embodiment shown in FIG. 3; however, FIG. 4 shows that the NPG production process can start at aldol condensation, rather than hydroformylation. In such a scenario, the isobutyraldehyde and/or r-isobutyraldehyde used in the aldol condensation reaction could be produced by a different entity and/or at a different site.

The recycled content NPG can be used in a variety of end use applications. For example, r-NPG can be used in hydraulic fluids, lubricants, greases, working fluids, textiles, pharmaceuticals, pesticides, and plasticizers. It may also be used as a monomer when forming polymers suitable in a wide variety of applications (e.g., polyesters for packaging, containers, shrink films, etc.) as well as base resins for coatings (e.g., polyurethanes).

### Claim Supporting Description—First Embodiment

In a first embodiment of the present technology there is provided a process for producing neopentyl glycol having recycled content, the process comprising: (a) hydroformylating a first propylene and a first syngas to thereby produce an isobutyraldehyde; (b) aldol condensing at least a portion of the isobutyraldehyde and a first formaldehyde to thereby produce a hydroxypivaldehyde; and (c) hydrogenating at least portion of the hydroxypivaldehyde with a first hydrogen to thereby produce a neopentyl glycol (NPG), wherein the NPG comprises recycled content from one or more of the following source materials—(i) a waste plastic, (ii) a recycled content syngas (r-syngas), (iii) a recycled content formaldehyde (r-formaldehyde), (iv) a recycled content hydrogen (r-H2), and/or (v) a recycled content propylene (r-propylene).

The first embodiment described in the preceding paragraph can also include one or more of the additional aspects listed below. The each of the following additional aspects of the first embodiment can be standalone features or can be combined with one or more of the other additional aspects to the extent consistent. Additionally, the following bullet pointed paragraphs can be viewed as dependent claim features having levels of dependency indicated by the degree of indention in the bulleted list (i.e., a feature indented further than the feature(s) listed above it is considered dependent on the feature(s) listed above it).
wherein the NPG comprises physical recycled content from one or more of the source materials.
wherein the NPG comprises credit-based recycled content from one or more of the source materials.
wherein the NPG comprises physical and credit-based recycled content from one or more of the source materials.
wherein the NPG comprises recycled content from the r-syngas.
   wherein the NPG has 5 to 59, 10 to 50, 20 to 40, or 25 to 35 percent recycled content from the r-syngas.
      wherein the NPG comprises physical recycled content from the r-syngas.
      wherein the NPG comprises credit-based recycled content from the r-syngas.
wherein the NPG comprises recycled content from the r-formaldehyde.
   wherein the NPG has 5 to 29, 10 to 29, 20 to 29, or 25 to 29 percent recycled content from the r-formaldehyde.
      wherein the NPG comprises physical recycled content from the r-formaldehyde.
      wherein the NPG comprises credit-based recycled content from the r-formaldehyde.
wherein the NPG comprises recycled content from the r-H2.
   wherein the NPG has 0.1 to 6, 0.5 to 5, 1 to 5, or 1.5 to 3 percent recycled content from the r-H2.
      wherein the NPG comprises physical recycled content from the r-H2.
      wherein the NPG comprises credit-based recycled content from the r-H2.
wherein the NPG comprises recycled content from the r-propylene.
   wherein the NPG has 10 to 40, 20 to 40, 30 to 50, or 35 to 45 percent recycled content from the r-propylene.
      wherein the NPG comprises physical recycled content from the r-propylene.
      wherein the NPG comprises credit-based recycled content from the r-propylene.
wherein the hydroformylating, aldol condensing, and hydrogenating are carried out in an NPG production facility, wherein at least one the source materials providing recycled content to the NPG is produced in a remote source facility that is located at least 0.5, 1, 5, 10, 100, 500, 1000 or 10,000 miles from the NPG production facility.
   wherein the NPG comprises credit-based recycled content from the at least one source material produced in the remote source facility.
wherein the hydroformylating, aldol condensing, and hydrogenating are carried out in an NPG production facility, wherein at least one of the source materials is produced in a co-located source facility located within 5, 1, 0.5, or 0.25 miles of the NPG production facility.
   wherein the NPG comprises physical recycled content from the at least on source material produced in the remote source facility.
further comprising making at least a portion of the r-syngas from at least one recycled content hydrocarbon-containing feedstock.
   wherein the making comprises partial oxidation gasification and/or steam reforming of the hydrocarbon-containing feedstock.
   wherein at least one of the following criteria is met—
      (i) the first syngas comprises at least a portion of the r-syngas,
      (ii) the first formaldehyde is formed from at least a portion of the r-syngas, and/or
      (iii) the first hydrogen comprises recycled hydrogen (r-H2) produced during the making of the r-syngas.
   wherein at least a portion of the first formaldehyde is produced by converting a second syngas into a methanol and then dehydrogenating at least a portion of the methanol.
      wherein the second syngas comprises at least a portion of the r-syngas
   wherein at least a portion of the r-H2 produced during the making of the r-syngas.
      wherein the first hydrogen comprises at least a portion of the r-H2.
wherein at least a portion of the r-propylene is made from a waste plastic.
   wherein at least a portion of the r-propylene is made by pyrolyzing and/or cracking the waste plastic.
   wherein the first propylene comprises at least a portion of the r-propylene.
comprising apply credit-based recycled content to the NPG from the one or more source materials.
   wherein the applying includes (i) attributing recycled content from at least one of the source materials having physical recycled content to at least one target material via recycled content credits, (ii) tracing recycled content along at least one chemical pathway from the at least one target material to the NPG, and (iii) allocating recycled content to the NPG based at least in part on the tracing of recycled content along the chemical pathway.
      wherein at least one of the following criterial is met (i) the source material and the target material both comprise syngas, (ii) the source material and the target material both comprise formaldehyde, (iii) the source material and the target material both comprise hydrogen, and/or (iv) the source material and the target material both comprise propylene.
         wherein the at least one target material comprise one or more of the first syngas, the first formaldehyde, the first hydrogen, and/or the first propylene.
      wherein at least one of the following criteria (i)-(iv) is met:
         (i) the source material comprises the r-syngas having physical recycled content, the target material comprises the first syngas, and the NPG has 5 to 59, 10 to 50, 20 to 40, or 25 to 35 percent credit-based recycled content from the r-syngas,
         (ii) the source material comprises the r-formaldehyde having physical recycled content, the target material comprises the first formaldehyde, and the NPG has 5 to 29, 10 to 29, 20 to 29, or 25 to 29 percent credit-based recycled content from the r-formaldehyde,
         (iii) the source material comprises the r-H2 having physical recycled content, the target material comprises the first hydrogen, and the NPG has 0.1 to 5.9, 0.5 to 20, 1 to 10, or 1.5 to 3 percent credit-based recycled content from the r-H2, and/or
         (iv) the source material comprises the r-propylene having physical recycled content, the target material comprises the first propylene, and the r-NPG has 10 to 40, 20 to 40, 30 to 50, or 35 to 45 percent credit-based recycled content from the r-propylene.
      wherein the attributing includes (i) booking recycled content credits attributable to the at least one source material into a digital inventory and (ii) assigning recycled content credits from the digital inventory to the target material.
         wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the NPG.

### Claim Supporting Description—Second Embodiment

In a second embodiment of the present technology there is provided a process for producing neopentyl glycol having recycled content, the process comprising: (a) aldol condensing a first isobutyraldehyde and a first formaldehyde to thereby produce a hydroxypivaldehyde; and (b) hydrogenating at least portion of the hydroxypivaldehyde with a first hydrogen to thereby produce a neopentyl glycol (NPG), wherein the NPG comprises recycled content from one or more of the following source materials—(i) a waste plastic, (ii) a recycled content formaldehyde (r-formaldehyde), and/or (iii) a recycled content hydrogen (r-H2).

The second embodiment described in the preceding paragraph can also include one or more of the additional aspects listed below. The each of the following additional aspects of the second embodiment can be standalone features or can be combined with one or more of the other additional aspects to the extent consistent.
wherein the first isobutyraldehyde comprises recycled content isobutyraldehyde.
wherein the first formaldehyde comprises the recycled content formaldehyde.
further comprising hydroformylating propylene and syngas to form an isobutyraldehyde, wherein at least one of the propylene and syngas have recycled content and the isobutyraldehyde comprises recycled content isobutyraldehyde (r-isobutyraldehyde).
further comprising synthesizing methanol from a first syngas and dehydrating at least a portion of the methanol to form at least a portion of the first formaldehyde used in the aldol condensing of step (a).
   further comprising carbon reforming a recycled content hydrocarbon-containing feed to provide a recycled content syngas (r-syngas), wherein the first syngas comprise the r-syngas and the first formaldehyde is an r-formaldehyde.
      wherein the recycled content hydrocarbon-containing feed comprises waste plastic.
      wherein the carbon reforming produces a recycled content hydrogen (r-H2), wherein the first hydrogen comprises at least a portion of the r-H2.
further comprising apply credit-based recycled content to the NPG from the one or more source materials.
   wherein the applying includes (i) attributing recycled content from at least one of the source materials having physical recycled content to at least one target material via recycled content credits, (ii) tracing recycled content along at least one chemical pathway from the at least one target material to the NPG, and (iii) allocating recycled content to the NPG based at least in part on the tracing of recycled content along the chemical pathway.
      wherein at least one of the following criterial is met (i) the source material and the target material both comprise syngas, (ii) the source material and the target material both comprise formaldehyde, (iii) the source material and the target material both comprise hydrogen, and/or (iv) the source material and the target material both comprise propylene.
         wherein the at least one target material comprise one or more of the first syngas, the first formaldehyde, the first hydrogen, and/or the first propylene.
      wherein at least one of the following criteria is met:
         (i) the source material comprises the r-formaldehyde having physical recycled content, the target material comprises the first formaldehyde, and the NPG has 5 to 59, 10 to 50, 20 to 40, or 25 to 35 percent credit-based recycled content from the r-formaldehyde, and/or
         (iii) the source material comprises the r-H2 having physical recycled content, the target material comprises the first hydrogen, and the NPG has 0.1 to 5.9, 0.5 to 20, 1 to 10, or 1.5 to 3 percent credit-based recycled content from the r-H2, and/or
      wherein the attributing includes (i) booking recycled content credits attributable to the at least one source material into a digital inventory and (ii) assigning recycled content credits from the digital inventory to the target material.
         wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the NPG.

### Claim Supporting Description—Third Embodiment

In a third embodiment of the present technology there is provided a process for producing formaldehyde having recycled content, the process comprising: (a) carbon reforming a hydrocarbon-containing feedstock to provide a first syngas; (b) synthesizing a first methanol from at least a portion of the first syngas; and (c) dehydrogenating at least a portion of the first methanol to provide formaldehyde, wherein the formaldehyde comprises recycled content from one or more of the following source materials—(i) a waste plastic, (ii) a recycled content hydrocarbon-containing feedstock (r-HC), (iii) a recycled content syngas (r-syngas), and/or (iv) a recycled content methanol (r-methanol).

The third embodiment described in the preceding paragraph can also include one or more of the additional aspects listed below. The each of the following additional aspects of the second embodiment can be standalone features or can be combined with one or more of the other additional aspects to the extent consistent.
wherein the formaldehyde comprises physical recycled content.
wherein the formaldehyde comprises credit-based recycled content.
wherein the hydrocarbon-containing feedstock comprises recycled content from waste plastic.
   wherein the hydrocarbon-containing feedstock comprises non-recycled content.
wherein the first methanol is synthesized from a second syngas, wherein said second syngas comprises r-syngas.
wherein the formaldehyde is formed by dehydrogenating a second methanol, wherein said second methanol comprises the r-methanol.
further comprising apply credit-based recycled content to the formaldehyde from the one or more source materials.
   wherein the applying includes (i) attributing recycled content from at least one of the source materials having physical recycled content to at least one target material via recycled content credits, (ii) tracing recycled content along at least one chemical pathway from the at least one target material to the formaldehyde, and (iii) allocating recycled content to the formaldehyde based at least in part on the tracing of recycled content along the chemical pathway.
      wherein the attributing includes (i) booking recycled content credits attributable to the at least one source material into a digital inventory and (ii) assigning recycled content credits from the digital inventory to the target material, and wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the formaldehyde.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and CO) that are useful by themselves and/or are useful as feedstocks to another chemical production process(es).

As used herein, the term “co-located” refers to the characteristic of at least two objects being situated on a common physical site, and/or within 5, 1, 0.5, or 0.25 miles of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycled content based on the mass of the recycled content in various materials.

As used herein, the terms “physical recycled content” and “direct recycled content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “recycled content” refers to being or comprising a composition that is directly and/or indirectly derived from recycle material. Recycled content is used generically to refer to both physical recycled content and credit-based recycled content. Recycled content is also used as an adjective to describe material having physical recycled content and/or credit-based recycled content.

As used herein, the term “recycled content credit” refers to a non-physical measure of physical recycled content that can be directly or indirectly (i.e., via a digital inventory) attributed from a first material having physical recycled content to a second material having less than 100 percent physical recycled content.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials.

### CLAIMS NOT LIMITED TO DISCLOSED EMBODIMENTS

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing neopentyl glycol having recycled content, the process comprising:
(a) hydroformylating a first propylene and a first syngas to thereby produce an isobutyraldehyde;
(b) aldol condensing at least a portion of the isobutyraldehyde and a first formaldehyde to thereby produce a hydroxypivaldehyde; and
(c) hydrogenating at least portion of the hydroxypivaldehyde with a first hydrogen to thereby produce a neopentyl glycol (NPG),
wherein the NPG comprises recycled content from one or more of the following source materials—(i) a waste plastic, (ii) a recycled content syngas (r-syngas), (iii) a recycled content formaldehyde (r-formaldehyde), (iv) a recycled content hydrogen (r-H2), and/or (v) a recycled content propylene (r-propylene).

2. The process of claim 1, wherein the NPG comprises physical recycled content from one or more of the source materials.

3. The process of claim 1, wherein the NPG comprises credit-based recycled content from one or more of the source materials.

4. The process of claim 1, wherein the NPG comprises 5 to 59 percent recycled content from the r-syngas.

5. The process of claim 1, wherein the NPG comprises 5 to 29 percent recycled content from the r-formaldehyde.

6. The process of claim 1, wherein the NPG comprises 0.1 to 6 percent recycled content from the r-H2.

7. The process of claim 1, wherein the NPG comprises 10 to 40 percent recycled content from the r-propylene.

8. The process of claim 1, wherein the hydroformylating, aldol condensing, and hydrogenating are carried out in an NPG production facility, wherein at least one the source materials providing recycled content to the NPG is produced in a remote source facility that is located at least 5 miles from the NPG production facility, and wherein the NPG comprises credit-based recycled content from the at least one source material produced in the remote source facility.

9. The process of claim 1, wherein the hydroformylating, aldol condensing, and hydrogenating are carried out in an NPG production facility, wherein at least one of the source materials is produced in a co-located source facility located within 5 miles of the NPG production facility, and wherein the NPG comprises physical recycled content from the at least one source material produced in the remote source facility.

10. The process of claim 1, further comprising making at least a portion of the r-syngas from at least one recycled content hydrocarbon-containing feedstock, wherein at least one of the following criteria (i) through (iii) is met—
(i) the first syngas comprises at least a portion of the r-syngas,
(ii) the first formaldehyde is formed from at least a portion of the r-syngas, and/or
(iii) the first hydrogen comprises recycled hydrogen (r-H2) produced during the making of the r-syngas.

11. The process of claim 10, wherein at least a portion of the first formaldehyde is produced by converting a second syngas into methanol and then dehydrogenating at least a portion of the methanol, and wherein the second syngas comprises at least a portion of the r-syngas.

12. The process of claim 10, wherein at least a portion of the r-H2 is produced during the making of the r-syngas and wherein the first hydrogen comprises at least a portion of the r-H2.

13. The process of claim 1, wherein at least a portion of the r-propylene is made by pyrolyzing and/or cracking waste plastic and wherein the first propylene comprises at least a portion of the r-propylene.

14. The process of claim 1, further comprising apply credit-based recycled content to the NPG from the one or more source materials, wherein the applying includes (i) attributing recycled content from at least one of the source materials having physical recycled content to at least one target material via recycled content credits, (ii) tracing recycled content along at least one chemical pathway from the at least one target material to the NPG, and (iii) allocating recycled content to the NPG based at least in part on the tracing of recycled content along the chemical pathway.

15. The process of claim 14, wherein at least one of the following criteria (i)-(iv) is met:
(i) the source material comprises the r-syngas having physical recycled content, the target material comprises the first syngas, and the NPG has 5 to 59 percent credit-based recycled content from the r-syngas;
(ii) the source material comprises the r-formaldehyde having physical recycled content, the target material comprises the first formaldehyde, and the NPG has 5 to 29 percent credit-based recycled content from the r-formaldehyde,
(iii) the source material comprises the r-H2 having physical recycled content, the target material comprises the first hydrogen, and the NPG has 0.1 to 6 percent credit-based recycled content from the r-H2, and/or
(iv) the source material comprises the r-propylene having physical recycled content, the target material comprises the first propylene, and the r-NPG has 10 to 40 percent credit-based recycled content from the r-propylene.

16. The process of claim 14, wherein the attributing includes (i) booking recycled content credits attributable to the at least one source material into a digital inventory and (ii) assigning recycled content credits from the digital inventory to the target material, wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the NPG.

17. A process for producing neopentyl glycol having recycled content, the process comprising:
(a) aldol condensing a first isobutyraldehyde and a first formaldehyde to thereby produce a hydroxypivaldehyde; and
(b) hydrogenating at least portion of the hydroxypivaldehyde with a first hydrogen to thereby produce a neopentyl glycol (NPG),
wherein the NPG comprises recycled content from one or more of the following source materials—(i) a waste plastic, (ii) a recycled content formaldehyde (r-formaldehyde), and/or (iii) a recycled content hydrogen (r-H2).

18. The process of claim 17, further comprising hydroformylating propylene and syngas to form an isobutyraldehyde, wherein at least one of the propylene and syngas have recycled content and the isobutyraldehyde comprises recycled content isobutyraldehyde (r-isobutyraldehyde).

19. The process of claim 17, further comprising synthesizing methanol from a first syngas and dehydrating at least a portion of the methanol to form at least a portion of the first formaldehyde used in the aldol condensing of step (a).

20. A process for producing formaldehyde having recycled content, the process comprising:
(a) carbon reforming a hydrocarbon-containing feedstock to provide a first syngas;
(b) synthesizing a first methanol from at least a portion of the first syngas; and
(c) dehydrogenating at least a portion of the first methanol to provide formaldehyde,
wherein the formaldehyde comprises recycled content from one or more of the following source materials—(i) a waste plastic, (ii) a recycled content hydrocarbon-containing feedstock (r-HC), (iii) a recycled content syngas (r-syngas), and/or (iv) a recycled content methanol (r-methanol).

21. The process of claim 21, wherein the first methanol is synthesized from a second syngas, wherein said second syngas comprises r-syngas and/or wherein the formaldehyde is formed by dehydrogenating a second methanol, wherein said second methanol comprises the r-methanol.

22. The process of claim 20, further comprising apply credit-based recycled content to the formaldehyde from the one or more source materials, wherein the applying includes (i) attributing recycled content from at least one of the source materials having physical recycled content to at least one target material via recycled content credits, (ii) tracing recycled content along at least one chemical pathway from the at least one target material to the formaldehyde, and (iii) allocating recycled content to the formaldehyde based at least in part on the tracing of recycled content along the chemical pathway.
